(19) United States
(12) Patent Application Publication
(10) Pub. No.: US 2023/0011056 A1
(43) Pub. Date: Jan. 12, 2023

(54) PRODUCT DISPENSING DEVICE

(71) Applicant: Nemera La Verpillière, LA VERPILLIERE (FR)

(72) Inventor: Alain Regard, Beynost (FR)

(21) Appl. No.: 17/784,949

(22) PCT Filed: Dec. 11, 2020
(86) PCT No.: PCT/EP2020/085849

§ 371 Date: Jun. 13, 2022

(30) Foreign Application Priority Data
Dec. 11, 2019 (FR) FR1914202

(51) Int. Cl. A61M 15/00

(52) U.S. Cl. CPC A61M 15/009; A61M 15/0021

(57) ABSTRACT

The invention relates to a product dispensing device configured to cooperate with a product reservoir that has product dispensing means, having a dispensing duct which is configured to dispense a product from the product dispensing means into the mouth of a user in a manner that is sealed against outside air, trigger means that trigger the dispensing of the product when the air pressure (Ai) in the dispensing duct is a predefined value greater than the pressure of the outside air (A). The invention further relates to a product administration kit having such a product dispensing device and to a product administration unit having such a product dispensing device.

### FIELD OF THE INVENTION

The invention relates to a product dispensing device.

### BACKGROUND OF THE INVENTION

The state of the art, as disclosed in document FR2954707, already describes an aerosol administration device that includes a mouthpiece used to administer an aerosol through the mouth during a nasal exhalation phase or during a respiratory pause phase preceding the nasal exhalation. In this device, the mouthpiece is airtight and extends beyond the patient's teeth over a certain length. Thus, in this case, the mouthpiece forms a means for administering the aerosol targeting the nasal, nasopharynx or nasosinusian region. The aerosol containing the particles is thus routed successively in the mouth, the nasopharynx and then the nasal cavities and the sinuses, until it escapes via at least one of the patient's nostrils. This document discloses in particular a device for administering an aerosol, the device forming a sealed circuit connected to the user's mouth comprising a mouthpiece, which allows inhalation and exhalation only through the nose. The user must manually trigger the administration of the aerosol during an inhalation phase according to a first variant, or during an exhalation phase according to a second variant. However, such manual triggering during an inhalation phase is complex and not practical for a user. In a third variant, the triggering is automatic but requires a nosepiece. However, such a nosepiece makes the aerosol administration device complex and more expensive, and makes the aerosol administration device less comfortable for a user, since it affects both the user's mouth and nose.

Thus, in view of their triggering, the current product dispensing devices used to dispense product into the mouth during an exhalation phase are complex, either as regards their use or as regards their structure.

### SUMMARY OF THE INVENTION

The invention aims in particular to provide a product dispensing device that is comfortable and easy to use for a user, and that proposes a simple triggering.

Thus, the invention relates to a product dispensing device configured to cooperate with a product reservoir provided with product dispensing means, comprising:

a dispensing duct which is configured to dispense a product from the product dispensing means into the mouth of a user in a manner that is sealed against outside air,

trigger means that trigger the dispensing of the product when the air pressure in the dispensing duct is a predefined value greater than the pressure of the outside air.

Thus, the invention proposes to trigger the dispensing of the product when the air pressure in the user's mouth is greater than the pressure of the outside air. It has been observed that the air pressure in the user's mouth is greater than the pressure of the outside air when the user is in a nasal exhalation phase, even in the absence of any extracorporeal element such as for example a nosepiece having a narrowing that reduces the cross-section through which the air is exhaled to the outside from the nose. This therefore ensures that the device only triggers if the user is in a nasal exhalation phase. Consequently, it is particularly comfortable and easy for the user to trigger the dispensing of product. It is understood that the fact of dispensing product during a nasal exhalation is particularly advantageous, since in this case the product can be dispensed from the mouth to the upper respiratory tracts, more precisely in the nasal, nasopharynx or nasosinusian region (to dispense the product in the sinuses, the nasopharynx or the nose), and not to the lower respiratory tracts such as the throat or the lung as it is the case when the product is dispensed when the user inhales.

The predefined value corresponds notably to the free nasal passages of a user and is therefore different from the overpressure values which would result from the presence of any extracorporeal element inserted in the nasal passages, which would reduce the cross-section through which the air is exhaled in the nasal passages.

The outside air is the ambient air, for example the air present around the user and the dispensing device. Thus, the outside air is not the air present in the mouth of a user, nor the air present in the dispensing duct or in the product reservoir, nor the product contained in the product reservoir.

According to other optional characteristics of the dispensing device taken alone or in combination:

the trigger means trigger the dispensing of the product when the user is in a nasal exhalation phase.

the dispensing duct is configured to cooperate, in a manner that is sealed against outside air, with a mouthpiece configured to lead into the user's mouth.

the predefined value is between 20 pascals and 500 pascals, preferably between 50 pascals and 150 pascals. Thus, the product dispensing device triggering threshold is optimum.

the trigger means comprise a movable element configured to move or deform elastically when the air pressure in the dispensing duct is greater than the pressure of the outside air. Thus, the use of a movable element provides a simple trigger means. This provides a simple way of detecting a pressure difference between the air present in the dispensing duct and the outside air.

the movable element is a membrane which deforms elastically when the air pressure in the dispensing duct is greater than the pressure of the outside air. Thus, the use of a membrane provides a purely mechanical, portable and independent, and therefore particularly simple, trigger means. It is therefore particularly easy to detect a pressure difference between the air present in the dispensing duct and the outside air, while preserving the sealing against the air outside the dispensing duct.

the product dispensing device further comprises stop means for the movable element, preferably for the membrane, which limit a displacement or an elastic deformation of the movable element when the air pressure in the dispensing duct is greater than the pressure of the outside air and/or when the air pressure in the dispensing duct is less than the pressure of the outside air. Thus, the displacement or deformation of the movable element, preferably the elastic deformation of the membrane, is limited to a maximum in one direction or the other, in particular when the user is in a nasal exhalation or inhalation phase. This provides a reusable product dispensing device. Preferably, this ensures that the membrane is only deformed elastically.

the trigger means comprise a switching mechanism which can be activated from an unstable balance position to a dispensing position, the switching mechanism being configured to cooperate with the product reservoir such that, when the switching mechanism is activated to the dispensing position, the product dispensing means dispense product. Thus, the use of a switching mechanism provides a particularly simple and inexpensive trigger means.

the switching mechanism cooperates with the movable element, preferably with the membrane, such that switching from the unstable balance position is caused by a displacement or a deformation of the movable element. Thus, the use of a switching mechanism cooperating with the movable element, preferably a membrane, provides a particularly simple and inexpensive trigger means that requires few parts and is therefore reactive.

the product dispensing device further comprises a casing configured to receive the product reservoir. Thus, the product dispensing device is compact and easy to use for the user.

the casing comprises pressing means configured to press on the product reservoir. Thus, the pressing means represent a simple way of providing energy to the dispensing means of the product reservoir when the trigger means trigger the dispensing of product. Product can then be effectively dispensed through the dispensing duct.

the switching mechanism comprises a drive lever, which pivots relative to the casing and is activated by the displacement or deformation of the movable element, and a triggering lever, which pivots relative to the casing and carries an arm, the arm pivoting relative to the triggering lever. Thus, the switching mechanism is made in a particularly simple way.

when the switching mechanism is in the unstable balance position, a free end of the arm is in contact with the drive lever. Thus, the unstable balance position of the switching mechanism is particularly easy to obtain.

the product dispensing device further comprises return means for returning the switching mechanism, which are configured to return the switching mechanism to the unstable balance position, preferably when the air pressure in the dispensing duct is less than or equal to the pressure of the outside air. Thus, the return means provide a reusable switching mechanism. Consequently, this provides a reusable product dispensing device.

the trigger means comprise a differential pressure sensor and an electromechanical trigger. Thus, the use of a differential pressure sensor and an electromechanical trigger provide a particularly compact and robust trigger means.

the trigger means are configured such that, when the sensor detects that the air pressure in the dispensing duct is a predefined value greater than the pressure of the outside air, a signal is transmitted to the electromechanical trigger which triggers the dispensing of product. Thus, accidental triggering is avoided.

The invention also relates to a product administration kit comprising a product dispensing device of the above-mentioned type, a mouthpiece, and optionally a product reservoir that has product dispensing means, in which the mouthpiece comprises a tube configured firstly to be connected to the dispensing duct in a manner that is sealed against outside air via a first end, and secondly to lead into the mouth of a user via the other end. Thus, such a kit allows easy transport for a simple subsequent assembly by a user of the dispensing device and of the mouthpiece, or even of the product reservoir.

The invention also relates to a product administration unit, which comprises a product dispensing device of the above-mentioned type, a mouthpiece, and optionally a product reservoir that has product dispensing means, in which:

the mouthpiece comprises a tube configured firstly to be connected to the dispensing duct in a manner that is sealed against outside air via a first end, and secondly to lead into the mouth of a user via the other end, and

the product dispensing means are configured to dispense product from the product reservoir towards the dispensing duct, when the product dispensing means are triggered by the trigger means.

According to other optional characteristics of the product administration unit taken alone or in combination:

the product reservoir is arranged removably in the casing. Thus, the unit is reusable, for example by replacing the product reservoir when it is empty.

the product reservoir is pressurized and sealed against outside air.

the elastic pressing means cooperate with reversible activation means to switch from a storage position, in which they do not press on the product reservoir, to an operating position, in which they press on the product reservoir. Unwanted triggering of dispensing is thus avoided.

the elastic pressing means are configured such that, when the switching mechanism is in the unstable balance position, they press, via the product reservoir, on the switching mechanism to hold it in the unstable balance position. Accidental triggering of dispensing is thus avoided.

the elastic pressing means are configured such that, when the switching mechanism is in the dispensing position, the elastic pressing means press on the product reservoir so that the product dispensing means dispense product from the product reservoir into the dispensing duct. Thus, the dispensing triggering is obtained in a particularly simple way.

### BRIEF DESCRIPTION OF THE DRAWINGS

The invention will be better understood on reading the following description, given solely by way of example and with reference to the accompanying drawings in which:

FIG. 1 is a schematic cross-sectional view of an administration unit comprising a dispensing device, in a storage position;

FIG. 2 is a schematic cross-sectional view of the administration unit shown on FIG. 1, in an operating position;

FIG. 3 is a schematic cross-sectional view of an administration unit shown on FIGS. 1 and 2, in a dispensing position;

FIG. 4 is a schematic perspective view of a part of the administration unit in the operating position shown on FIG. 2;

FIG. 5 is a schematic perspective view of a part of the administration unit in the dispensing position shown on FIG. 3.

### DETAILED DESCRIPTION OF THE INVENTION

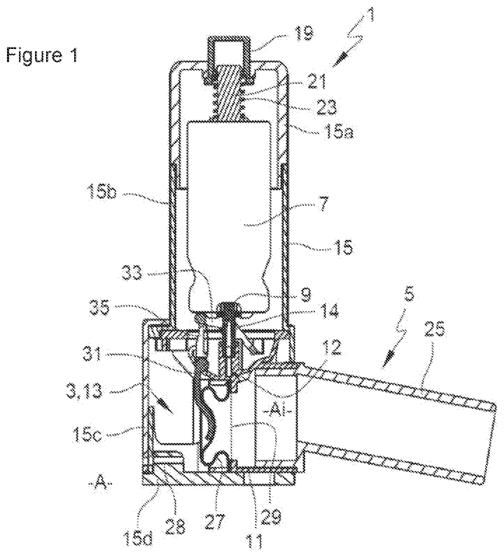

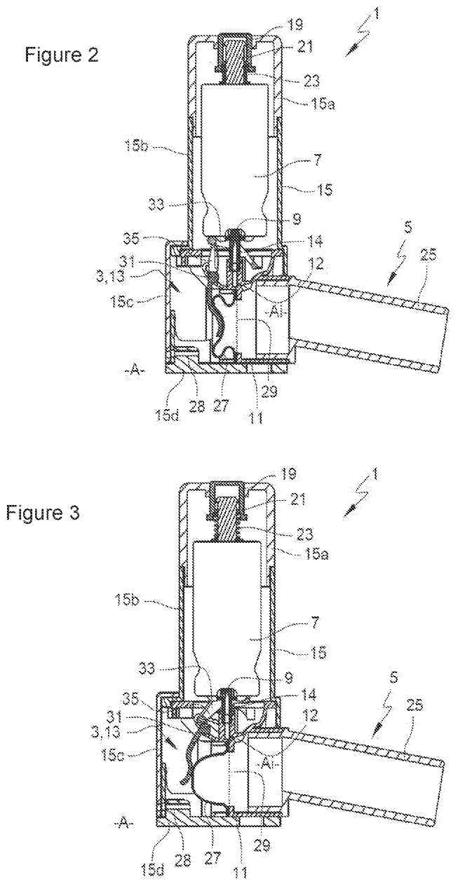

FIGS. 1 to 5 show a product administration unit according to the invention, designated by the general reference 1.

This product administration unit 1 comprises a product dispensing device 3, a mouthpiece 5 and a product reservoir 7 that has product dispensing means 9.

The product dispensing device 3 is configured to cooperate with the product reservoir 7 that has product dispensing means 9.

As shown on FIGS. 1 to 3, the product dispensing device 3 comprises a dispensing duct 11 which is configured to dispense a product from the product dispensing means 9 into the mouth of a user in a manner that is sealed against outside air A. The dispensing duct 11 is configured to cooperate, in a manner that is sealed against outside air A, with the mouthpiece 5 configured to lead into the user's mouth. The dispensing duct 11 is therefore designed to dispense the product, for example an aerosol. In the example shown, the dispensing duct 11 comprises a nozzle 12 which optimizes the distribution of product in the dispensing duct 11 in order to dispense it towards the mouth of a user.

The product dispensing device 3 also comprises trigger means 13 which trigger the dispensing of product when the air pressure Ai in the dispensing duct 11 is a predefined value greater than the pressure of the outside air A.

The predefined value is between 20 Pascals and 500 Pascals, preferably between 50 Pascals and 150 Pascals.

In the example shown, the trigger means 13 are configured to trigger the dispensing of the product when the user is in a nasal exhalation phase.

The product dispensing means 9 are configured to dispense product from the product reservoir 7 towards the dispensing duct 11, when the product dispensing means 9 are triggered by the trigger means 13. In the example shown, the product dispensing means 9 comprise a dispensing tube 14 having a distal end which is introduced into the dispensing duct 11, its other end being, for example, connected to a valve or a metered pump arranged in the product reservoir 7. The rest of the product reservoir 7 can slide relative to the dispensing tube 14, between an operating position, in which the product dispensing means 9 do not dispense product, and a dispensing position, in which the product dispensing means 9 dispense product out of the product reservoir 7 via the dispensing tube 14. The product reservoir 7 is, for example, pressurized and sealed against outside air A.

The product dispensing device 3 further comprises a casing 15 configured to receive the product reservoir 7. In the example shown, as illustrated for example on FIG. 1, the casing 15 consists of several casing sub-parts 15a, 15b, 15c, 15d, which are assembled to each other so as to be able to house the product reservoir 7 and the product dispensing device 3 inside the casing 15. In the example show, the product reservoir 7 is thus arranged removably in the casing 15, since the casing sub-part 15a, arranged in the upper part, is made to form a removable cover and can be disconnected from the casing sub-part 15b. The casing sub-part 15b is preferably at least partially transparent, so that a label affixed to the product reservoir 7 can be read.

In the example shown on FIGS. 1 to 3, the casing 15 comprises elastic pressing means 21, 23 configured to press on the product reservoir 7. These elastic pressing means comprise a rod 21 and a spring 23, and cooperate with reversible activation means 19. The reversible activation means 19 comprise, for example, an activation button 19. Thus, the elastic pressing means 21, 23 cooperate with the reversible activation means 19 to switch from a storage position, in which they do not press on the product reservoir 7, to an operating position, in which they press on the product reservoir 7. Thus, the elastic pressing means 21, 23 and the reversible activation means 19 make it possible to switch the product administration unit 1 respectively from a storage position to an operating position, from the operating position to a dispensing position, and from the dispensing position to the storage position. In the example shown, the elastic pressing means 21, 23 and the reversible activation means 19 are carried by the casing sub-part 15a.

The mouthpiece 5 comprises a pipe 25 configured firstly to be connected to the dispensing duct 11 in a manner that is sealed against outside air A via a first end, and secondly to lead into the mouth of a user via its other end.

The trigger means 13 comprise a movable element configured to move or deform elastically when the air pressure Ai in the dispensing duct 11 is greater than the pressure of the outside air A. In the example shown, the movable element is a membrane 27 which deforms elastically when the air pressure Ai in the dispensing duct is greater than the pressure of the outside air A. The membrane 27 is solidly attached to the dispensing duct 11 in a manner that is sealed against outside air, in other words, the outside air A does not pass through either the membrane 27, or the connection area between the membrane 27 and the dispensing duct 11. The membrane 27 is arranged so as to block an opening of the dispensing duct 11, so that an inner face of the membrane 27 is in contact with the air Ai in the dispensing duct 11. Another face of the membrane 27, referred to as the outer face, is in contact with the outside air A, thanks to the fact that the casing 15 comprises a passing hole 28 for the outside air A leading to an inner space of the casing 15 at least partially delimited by the outer face of the membrane 27.

As an alternative or in addition, the trigger means 13 comprise a differential pressure sensor and an electromechanical trigger. In this case, the trigger means 13 are configured such that, when the sensor detects that the air pressure Ai in the dispensing duct 11 is a predefined value greater than the pressure of the outside air A, a signal is transmitted to the electromechanical trigger which moves and triggers the dispensing of product.

The product dispensing device 3 further comprises stop means for the movable element, preferably for the membrane 27, which limit a displacement or an elastic deformation of the movable element when the air pressure Ai in the dispensing duct is greater than the pressure of the outside air A and/or when the air pressure Ai in the dispensing duct 11 is less than the pressure of the outside air A. In the example shown, these stop means consist, for example, of a grid 29 arranged on the opening of the dispensing duct 11 blocked by the membrane 27, which limits the deformation of the membrane 27 when the air pressure Ai in the dispensing duct 11 is less than the pressure of the outside air A.

As shown on the figures, the trigger means 13 comprise a switching mechanism 31, 33 which can be activated from an unstable balance position to a dispensing position, the switching mechanism 31, 33 being configured to cooperate with the product reservoir 7 such that, when the switching mechanism 31, 33 is activated to the dispensing position, the product dispensing means 9 dispense product. The switching mechanism 31, 33 cooperates with the movable element, for example the membrane 27, such that switching from the unstable balance position to the dispensing position is caused by a displacement or a deformation of the movable element.

The switching mechanism 31, 33 comprises a drive lever 31, which pivots relative to the casing 15 and is activated by the displacement or deformation of the movable element, in this case the membrane 27. In this example, the drive lever 31 is in permanent contact with the membrane 27, this permanent contact being kept for example under the action of gravity on the drive lever 31, by a mechanical link or by bonding.

The switching mechanism 31, 33 also comprises a triggering lever 33 which pivots relative to the casing 15 and further carries an arm 35, the arm 35 pivoting relative to the triggering lever 33. The arm 35 is arranged such that when the switching mechanism 31, 33 is in the unstable balance position, a free end of the arm 35 is in contact with the drive lever 31, preferably at a pivot of the drive lever 31 pivoting relative to the casing 15.

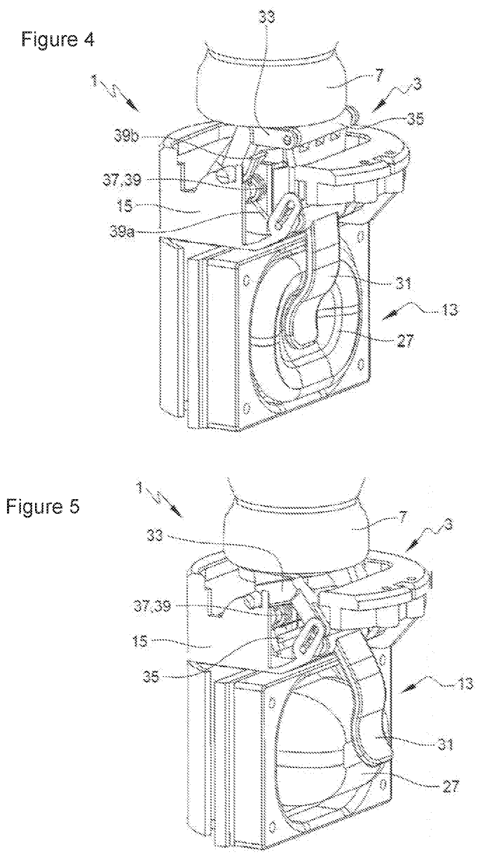

As shown on FIG. 4, the product dispensing device 3 further comprises return means 37 for returning the switching mechanism 31, 33, which are configured to return the switching mechanism 31, 33 to the unstable balance position, preferably when the air pressure Ai in the dispensing duct 11 is less than or equal to the pressure of the outside air A, and more preferably, when the elastic pressing means 21, 23 and the reversible activation means 19 no longer cooperate and are in the storage position. In the example show, these return means 37 comprise a torsion spring 39 arranged around a trunnion rigidly attached to the casing 15. The torsion spring 39 comprises an end 39a pressing on the casing 15, and another end 39b pressing on the triggering lever 33.

Furthermore, the elastic pressing means 21, 23 are configured such that, when the switching mechanism 31, 33 is in the unstable balance position, they press, via the product reservoir 7, on the switching mechanism 31, 33 to hold it in the unstable balance position. The product reservoir 7 in fact presses its inner face on the side of the dispensing tube 14 on one end of the triggering lever 33, said end carrying the arm 35. The arm 35 is arranged so as to pivot at one of its two ends relative to the triggering lever and is in contact, at its opposite end, with the drive lever 31. Thus, the triggering lever 33 presses on the arm 35 which itself presses on the drive lever 31. The elastic pressing means 21, 23 are also configured such that, when the switching mechanism 31, 33 is in the dispensing position, the elastic pressing means 21, 23 press on the product reservoir 7 so that the product dispensing means 9 dispense product from the product reservoir 7 into the dispensing duct 11, for example by the product reservoir 7 sliding relative to the dispensing tube 14.

The switch from the unstable balance position to the dispensing position takes place as follows. When the air pressure Ai in the dispensing duct 11 is greater than the pressure of the outside air A, the membrane 27 deforms and drives in rotation the drive lever 31 about its pivoting axis relative to the casing 15. Due to this rotation, the lower end of the arm 35 in contact with the drive lever 31 is moved, and the arm 35 pivots relative to the triggering lever 33. The unstable balance position is then lost, and the rod 21 presses on the dispensing reservoir 7 under the effect of the spring 23 to make it slide relative to the dispensing tube 14. Because the dispensing reservoir 7 presses on the triggering lever, it pivots the triggering lever 33 relative to the casing 15. The end of the triggering lever 33 carrying the arm 35 moves down and then drives the arm 35 such that the arm 35 slides against the drive lever 31. The dispensing position is then reached, and product can be dispensed from the product reservoir 7 through the dispensing tube 14, then through the dispensing duct 11 and the pipe 25 towards the user's mouth.

The elements of the product administration unit 1 may consist of at least one polymer material. They can be manufactured using an injection moulding process.

The spring 23 and the torsion spring 39 are for example made of steel, preferably stainless steel or equivalent known by those skilled in the art. The product reservoir 7 is a component known by those skilled in the art, and can be a standard component consisting of a pressurized product storage reservoir, the product reservoir 7 comprising a pot's shape metal wall, which is sealed by a metal ferrule, from which the dispensing tube 14 of the product dispensing means 9 comes out. The dispensing tube 14 is made for example of at least one polymer material. The membrane 27 is for example made of an elastomer material, preferably with a Shore A hardness between 40 and 50, such as for example a thermoplastic elastomer or a silicone. The membrane 27 is for example 0.4 millimetres thick.

A product administration kit is also described. Such a product administration kit is for example manufactured such that it comprises a product dispensing device 3 as previously described, a mouthpiece 5 for example as previously described, and optionally a product reservoir 7 for example as previously described, that has product dispensing means 9 for example as previously described. For example, the mouthpiece 5 comprises a pipe 25 configured firstly to be connected to the dispensing duct 11 in a manner that is sealed against outside air via a first end, and secondly to lead into the mouth of a user via its other end.

A method for dispensing a product using a product administration unit 1 as described previously is also described.

Such a method comprises at least the following steps:

pressing on the activation button 19 by a user. Thus, the activation button 19 compresses the spring 21 arranged between the activation button 19 and the rod 21, which causes the rod 21 to press on the product reservoir 7. The product administration unit 1 is then in an operating position. The reservoir 7 presses on the triggering lever 33, which itself presses on the arm 35, the latter pressing on the drive lever 31. The switching mechanism 31, 33 is then in the unstable balance position.

inserting the free end of the mouthpiece into the user's mouth.

exhaling by the user through the nasal passages, which triggers the trigger means 13 and activates the switching mechanism 31, 33 to the dispensing position.

dispensing product from the product reservoir 7 through the dispensing tube 14, then through the dispensing duct 11, then through the pipe 25 towards the user's mouth.

The invention is not limited to the examples described and other embodiments will be clearly apparent to those skilled in the art. Instead of a grid 19, a simple transverse bar could be used.

### LIST OF REFERENCES

1: product administration unit
3: product dispensing device
5: mouthpiece
7: product reservoir
9: product dispensing means
11: dispensing duct
13: trigger means
14: dispensing tube
15: casing
15a, 15b, 15c, 15d: casing sub-parts
19: reversible activation means, activation button
21, 23: elastic pressing means
21: rod
23: spring
25: pipe
27: membrane
29: grid
31, 33: switching mechanism
31: drive lever
33: triggering lever
35: arm
37: return means
39: torsion spring
39a, 39b: ends of the torsion spring
A: outside air
Ai: air (in the dispensing duct)

1. A product dispensing device configured to cooperate with a product reservoir provided with a product dispensing unit, comprising:
a dispensing duct configured to dispense a product from the product dispensing unit into a mouth of a user in a manner that is sealed against outside air,
a trigger unit that triggers the dispensing of the product when an air pressure (Ai) in the dispensing duct is a predefined value greater than a pressure of the outside air (A).

2. The product dispensing device according to claim 1, wherein the trigger unit comprises a movable element configured to move or deform elastically when the air pressure (Ai) in the dispensing duct is greater than the pressure of the outside air (A).

3. The product dispensing device according to claim 2, wherein the movable element is a membrane, which deforms elastically when the air pressure (Ai) in the dispensing duct is greater than the pressure of the outside air (A).

4. The product dispensing device according to claim 1, wherein the trigger unit comprises:
a switching mechanism configured to activated from an unstable balance position to a dispensing position, the switching mechanism being configured to cooperate with the product reservoir such that, when the switching Page 5 mechanism is activated to the dispensing position, the product dispensing unit dispenses product.

5. The product dispensing device according to 4, wherein the switching mechanism cooperates with the movable element such that switching from the unstable balance position is caused by a displacement or a deformation of the movable element.

6. The product dispensing device according to claim 1, wherein it further comprises a casing configured to receive the product reservoir.

7. The product dispensing device according to claim 6, wherein the switching mechanism comprises:
a drive lever which pivots relative to the casing and is activated by the displacement or deformation of the movable element,
a triggering lever, which pivots relative to the casing and also carries an arm, the arm pivoting relative to the triggering lever.

8. The product dispensing device according to claim 7, wherein, when the switching mechanism is in the unstable balance position, a free end of the arm is in contact with the drive lever.

9. The product dispensing device according to claim 4, further comprising a return member for returning the switching mechanism, the return member configured to return the switching mechanism to the unstable balance position when air pressure (Ai) in the dispensing duct is less than or equal to the pressure of the outside air (A).

10. The product dispensing device according to claim 1, wherein the trigger comprises a differential pressure sensor and an electromechanical trigger.

11. A product administration kit comprising the product dispensing device according to claim 1, a mouthpiece, and a product reservoir that has the product dispensing unit,
wherein the mouthpiece comprises a pipe configured to be connected to the dispensing duct in a manner that is sealed against outside air (A) via a first end, and to lead into the mouth of a user via its other end.

12. A product administration unit comprising a product dispensing device according to claim 1, a mouthpiece and a product reservoir that has the product dispensing unit, wherein:
the mouthpiece comprises a pipe, configured to be connected to the dispensing duct in a manner that is sealed against outside air (A) via a first end, and to lead into the mouth of a user via the other end, and
the product dispensing unit is configured to dispense product from the product reservoir towards the dispensing duct when the product dispensing unit is triggered by the trigger unit.
